(19) United States
(12) Patent Application Publication
(10) Pub. No.: US 2012/0129252 A1
(43) Pub. Date: May 24, 2012

(54) METHOD AND SYSTEM FOR CELL FILTRATION

(76) Inventor: Ronald C. Seubert, City of Sammamish, WA (US)

(21) Appl. No.: 13/296,082

(22) Filed: Nov. 14, 2011

Related U.S. Application Data
(60) Provisional application No. 61/412,741, filed on Nov. 11, 2010.

(51) Int. Cl. C12N 5/10; B01D 69/02; B01D 63/00; C12M 1/12

(52) U.S. Cl. 435/325; 435/283.1; 210/500.24; 210/321.62

(57) ABSTRACT

Methods and systems disclosed in the present application include membrane-like filters and methods and systems that employ these membrane-like filters to isolate circulating tumor cells and other abnormal cells from biological fluids, such as blood. The disclosed methods and systems use membrane-like filters that include a pattern or array of small, tapered apertures fabricated within a relatively thin but mechanically robust polymeric material that resists accumulation of biological-solution components and clogging during filtration of biological solutions.

### CROSS-REFERENCE TO RELATED APPLICATION

This application claims the benefit of Provisional Application No. 61/412,741, filed Nov. 11, 2010.

### TECHNICAL FIELD

The present application is related to the analysis of blood and other biological fluids and, in particular, to a membrane-filtration method and system for separating circulating tumor cells and other particular types of cells from blood cells and other such components of biological solutions.

### BACKGROUND

Enormous research efforts have been expended, over the past 60 years, to understand and develop effective treatment and preventative measures for various types of cell-proliferation diseases generally referred to as “cancer.” While great progress has been made in many areas and facets of this complex scientific problem, and while, in certain cases, dramatic improvements in treatment of certain types of cancers has been developed, cancer remains one of the leading causes of death, particularly in older populations, and treatment of cancer still accounts for a very large proportion of total expenditures on health care.

The various types of cancers are complex diseases that manifest themselves in unchecked cell proliferation and the spread of cell proliferation, including the spread of tumor sites, throughout an organism. In the case of localized proliferative tissues, referred to as “tumors,” the process by which cell proliferation spreads throughout an organism is referred to as “metastasis.” With the advent of high-throughput genomic analysis and characterization of the information-containing molecules of tissues and methods for identification of genetic, metabolic, and other physiological changes in cells that lead to cancer, rapid progress is being made in understanding how various types of cancer arise and progress. However, research techniques directed to understanding the molecular biology and cell biology of various types of cancer are often expensive, involve significant time periods for analysis, are often carried out after the particular cancer has progressed to a fatal disease, and these methods are often carried out on tissues obtained from deceased patients. Diagnosticians and clinical personnel involved in diagnosing and treating cancer continue to seek methods for detecting cancer and monitoring the progression of cancer within patients in order to apply treatments to slow or prevent progression of various types of cancer to debilitating and fatal stages.

### SUMMARY

Methods and systems disclosed in the present application include membrane-like filters and methods and systems that employ these membrane-like filters to isolate circulating tumor cells and other abnormal cells from biological fluids, such as blood. The disclosed methods and systems use membrane-like filters that include a pattern or array of small, tapered apertures fabricated within a relatively thin but mechanically robust polymeric material that resists accumulation of biological-solution components and clogging during filtration of biological solutions.

### BRIEF DESCRIPTION OF THE DRAWINGS

FIG. 1 illustrates a simple filtration system that can be used, with a membrane-like filter that represents one embodiment of the present application, as a filtration system for filtering and isolating circulating tumor cells from blood, as well as for filtering and isolating other types of cells from blood and other types of biological substances.

FIG. 2 shows an expanded illustration of the lower fitting of the glass cylinder, the complementary fitting of the hollow adaptor, and a disk-shaped membrane-like filter that is clamped between the two fittings in the apparatus shown in FIG. 1.

FIG. 3 illustrates the chemical structure of PEEK.

FIGS. 4A-C illustrate one embodiment of a membrane-like filter for isolating circulating tumor cells.

FIGS. 5A-B illustrate a micromachined aperture within an array of micromachined apertures of a membrane-like filter used for isolating circulating tumor cells.

FIGS. 6A-C illustrate another type of filter housing that can be employed, along with a membrane-like filter with tapered apertures, to extract circulating tumor cells (“CTCs”) from blood and other biological fluid.

FIGS. 7A-B illustrate yet another type of system for supporting the membrane-like filter within a housing to facilitate passing a biological fluid or other CTC-containing fluid through the filter.

### DETAILED DESCRIPTION

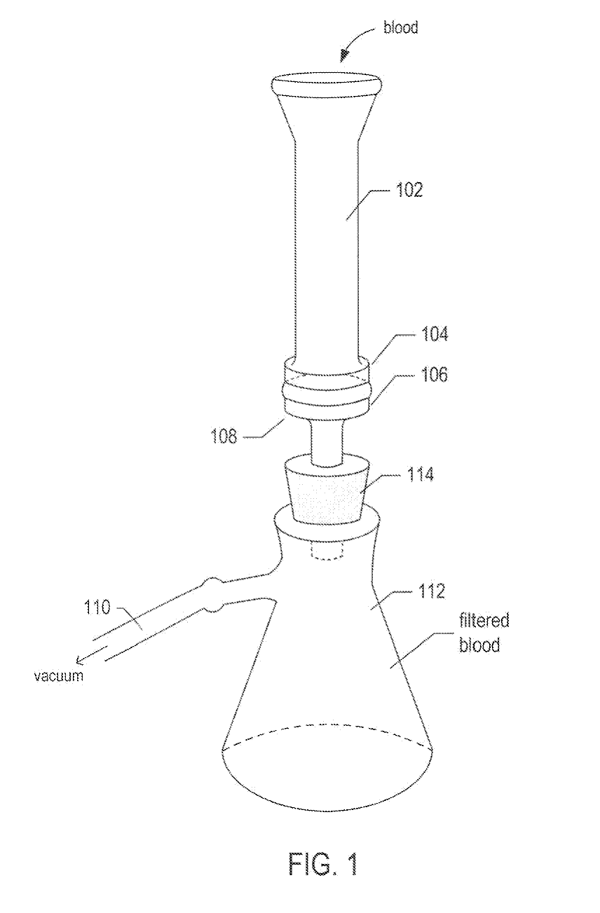

FIG. 1 illustrates a simple filtration system that can be used, with a membrane-like filter disclosed, in the current application, as a filtration system for filtering and isolating circulating tumor cells from blood, as well as for filtering and isolating other types of cells from blood and other types of biological substances. The biological substance is poured or dripped into a cylindrical glass column 102. The glass column 102 has a lower, ground-glass fitting 104 that mates with a similar fitting 106 of a lower, hollow cylindrical adaptor 108. A disk-shaped membrane-like filter is placed at the junction of the fitting 104 of the glass column 102 and the fitting 106 of the adaptor 108, and the adaptor and cylinder are clamped together to seal the adaptor and glass cylinder and form a single fluid chamber from the mouth of the glass cylinder to the end of the cylindrical adapter, with the membrane-like filter blocking flow of the biological solution from the glass cylinder 102 to the cylindrical adaptor 108.

When a relatively modest vacuum is applied to a tube 110 mounted to a distillation flask 112 to which the glass cylinder and adaptor are mounted via rubber stopper 114, the biological solution is pulled from the glass cylinder through the membrane-like filter into the flask 112. Because circulating tumor cells (“CTCs”) are larger than, and differently shaped from, red blood cells and other cells found in blood, the CTCs remain on the glass-cylinder side of the membrane-like filter and the non-CTC cells and other solution components pass through the membrane-like filter into the flask. Following filtration of the biological solution, the filter can then be removed from the apparatus, the CTC cells can be stained for greater visibility and contrast by various staining methods, and the filter can be examined under a microscope to identify, and count, and characterize the CTCs. Alternatively, the CTCs can be flushed from the filer into an analytical solution that can then be analyzed to count and characterize CTCs by various methods. By this relatively inexpensive, robust, and easily carried-out procedure, the presence and concentration of CTCs in blood samples can be readily determined in diagnostic and clinical settings. Membrane-like filters for filtering and isolating CTCs can be incorporated into automated analysis instruments in clinical laboratories for automated analysis of many different sample solutions in parallel. In such automated instrumentation, the CTCs may be isolated in a first filtration step, flushed from the membrane-like filter into an analytical solution in a second step, and then automatically flushed and cleaned in preparation for a next sample-solution analysis.

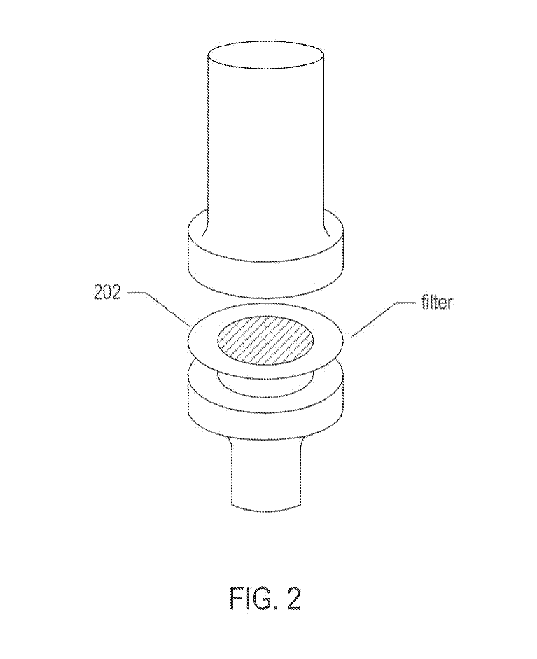

FIG. 2 shows an expanded illustration of the lower fitting of the glass cylinder, the complementary fitting of the hollow adaptor, and a disk-shaped membrane-like filter that is clamped between the two linings in the apparatus shown in FIG. 1. A wide variety of filter housings, supports, and scaling systems can be employed to mount filters 202 across flow channels in a variety of different types of filtration devices and systems.

The polymeric material employed to fabricate the membrane-like filters may determine the suitability and applicability of the filter to various types of analytic procedures and to various types of biological solutions. Many different types of polymeric materials have been tried in various types of membrane-like filters over the years, including polyethylene, parylene, and other types of polymers. However, these previously tried polymeric materials have proved unsuitable for various reasons. In certain cases, the polymeric materials do not provide sufficient mechanical strength and resistance to wear and damage and, in other cases, or additionally, the polymeric material may be susceptible to accumulation of biological substances during filtration and to clogging of micropores.

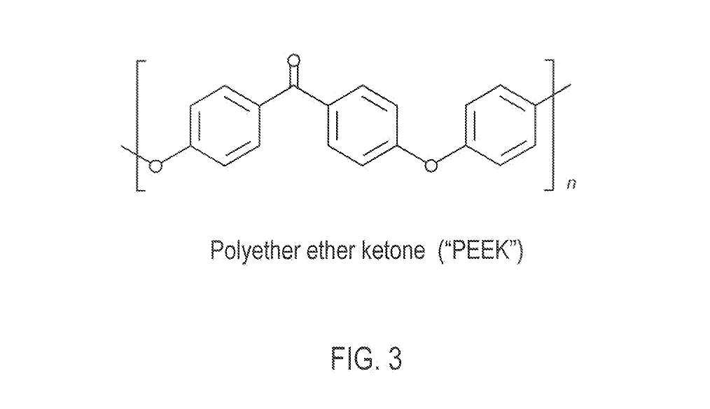

Certain embodiments of the present application employ the polymer polyether ether ketone (“PEEK”) for the membrane-like filter. FIG. 3 illustrates the chemical structure of PEEK. PEEK filters are tear-resistant and wear-resistant, can be micromachined precisely to create precisely defined microscale apertures, are highly resistant to the accumulation of biological tissues, materials, and other solution components during filtration procedures, and resist clogging. Alternative embodiments of the present application include filters manufactured from other types of polymers that resist accumulation of biological tissues, materials, and other solution components during filtration procedures, that resist clogging, and that provide sufficient mechanical strength for particular filtering applications.

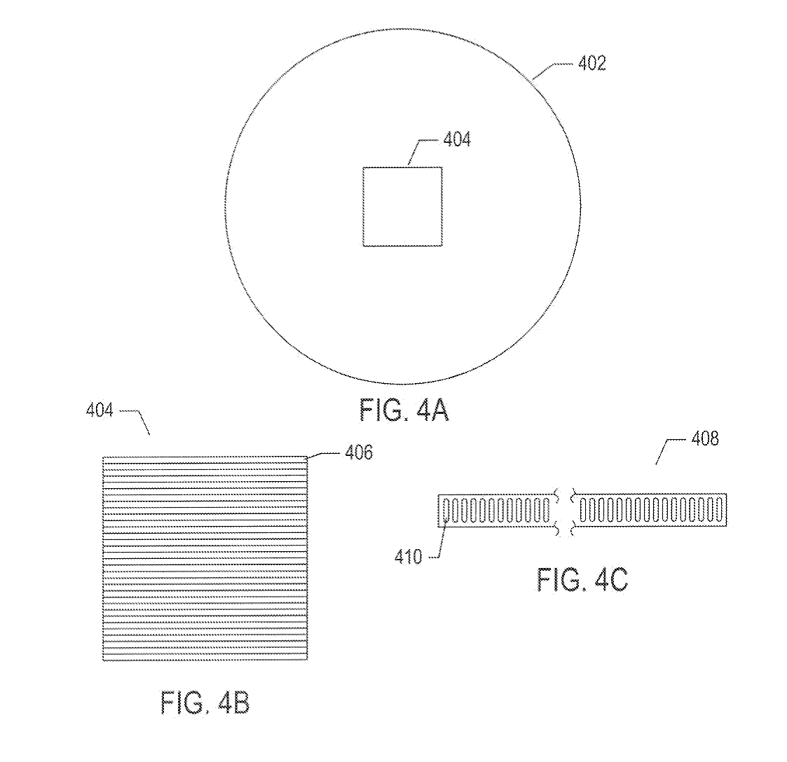

FIGS. 4A-C illustrate one embodiment of a membrane-like filter for isolating circulating tumor cells. FIG. 4A shows the disk-shaped membrane-like filter 402 also shown as filter 202 in FIG. 2. The disk-shaped filter comprises a PEEK film with a central array 404 of microscale apertures. FIG. 4B provides a larger-scale, more detailed illustration of the array of microscale apertures 404 shown in FIG. 4A. The array of microscale apertures 404 comprises a large number of rows, such as row 406, of regular micro-machined apertures. FIG. 4C shows, at larger scale, one of the rows of the microscale-aperture array 404 shown in FIGS. 4A-B. The row 408 includes a sequence of regularly spaced and regularly shaped and sized apertures, such as aperture 410.

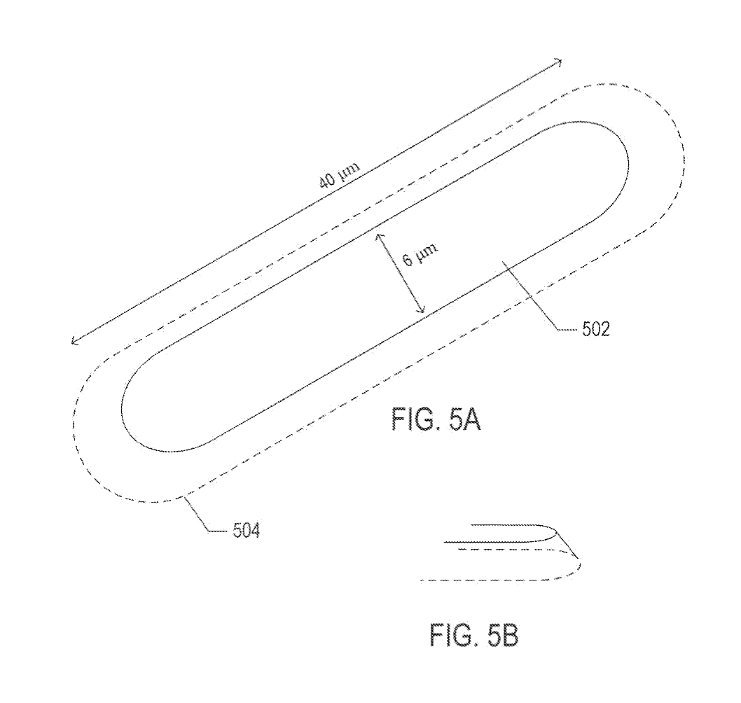

FIGS. 5A-B illustrate a micromachined aperture within an array of micromachined apertures of a membrane-like filter used for isolating circulating tumor cells. As shown in FIG. 5A, each micromachined aperture is a slot-like aperture 502, in one embodiment having a width of six μm and a length of 40 μm. The micromachined aperture is tapered, as indicated in FIG. 5A by the dashed outline 504 that represents the opening of the aperture on a lower surface of the membrane-like filter, while the solid-line aperture 502 represents the top opening of the micromachined aperture on the top surface of the membrane-like filter. The taper of the micromachined aperture is alternatively illustrated in FIG. 5B. The smaller-dimensioned opening of the aperture resides on the top of the filter exposed to the biological solution that is analyzed, while the larger-dimensioned opening is at the bottom of the membrane-like filter is positioned adjacent to the flask or other receptacle or chamber into which filtered biological solution passes. Because of the taper, it is unlikely or impossible for blood cells and other biological-solution components that pass through the aperture to accumulate and clog the aperture.

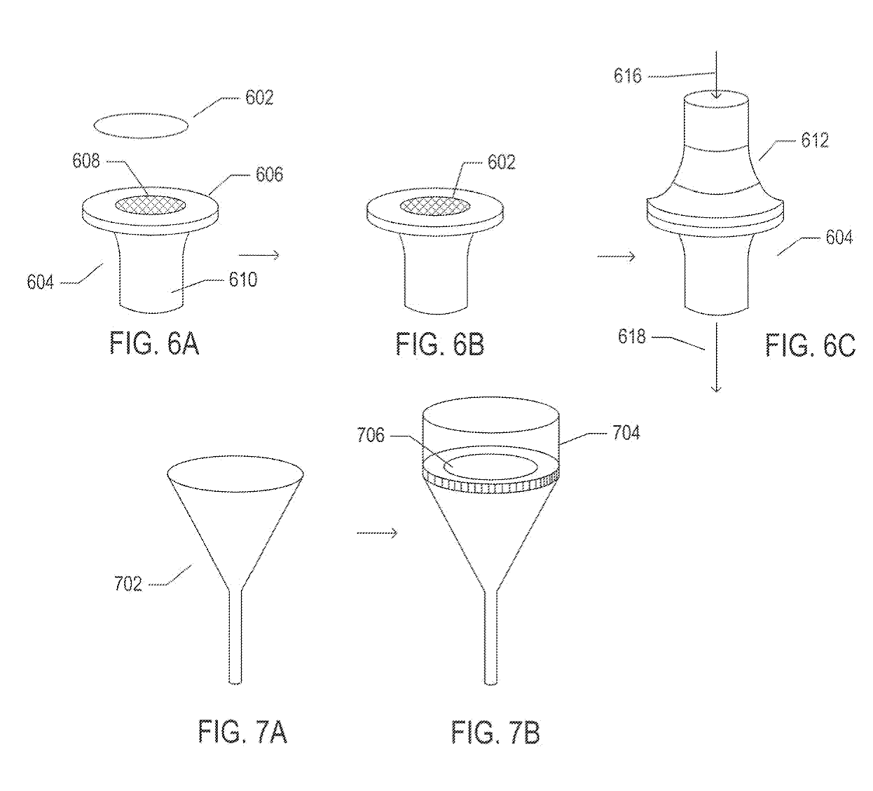

FIGS. 6A-C illustrate another type of filter housing that can be employed, along with a membrane-like filter with tapered apertures, to extract CTCs from blood and other biological fluids. FIG. 6A shows the membrane-like filter 602 positioned above a lower filter-housing component 604. The lower filter-housing component includes a disk-like platform 606, approximately normal to the symmetry axis, in which a mesh, grating, array of perforations, or other porous support 608 has been machined or otherwise fashioned to provide fluid communication from the region above the upper surface of the disk-shaped support platform 606 to a hollow interior channel within a stem 610 extending along the symmetry axis below the support. As shown in FIG. 6B, the membrane-like filter 602 is laid onto the porous support and, as shown in FIG. 6C, an upper filter-housing component 612 is joined to the lower filter-housing component 604 to form a fluid-impermeable annular seal enclosing the membrane-like filter within an interior volume formed by the joined filter-housing components. The two-component filter housing includes an upper tubular stem leading to the membrane-like filter/porous-support structure and a lower tube-like stem to which CTC-containing fluid can be pushed, in the direction of arrows 616 and 618, by hydrostatic pressure or pumping, resulting in filtration of the CTCs, with the CTCs remaining on the upper surface of the membrane-like filter. Alternatively, the biological or other CTC-containing fluid may be pulled through the filter housing and membrane-like filter from below by vacuum suction or surface-tension-based siphoning. The two components of the filter housing are secured, in place, by one or more clamps, a thin bead of sealant, and/or by any of various other securing means and combinations of securing means. In general, the support 608 includes apertures with diameters or areas larger than the corresponding diameters or areas of the lower openings of the tapered apertures of the membrane-like filter, but small enough to adequately support the membrane-like filter in order to prevent tearing or distortion of the membrane-like filter when pressure is applied to drive the biological fluid or other CTC-containing fluid through the membrane-like filter.

FIGS. 7A-B illustrate yet another type of system for supporting the membrane-like filter within a housing to facilitate passing a biological fluid or other CTC-containing fluid through the filter. As shown in FIG. 7A, the lower portion of this alternative housing is a glass or plastic funnel 702 or a funnel made from another rigid material impermeable to an aqueous medium. As shown in FIG. 7B, a cylindrical housing 704 is mounted to the open end of the funnel, the cylindrical housing including a support (not shown in FIG. 7B), similar to support 608 in FIG. 6A, above which the membrane-like filter 706 has been positioned. In certain cases, the membrane-like filter may be securely held in place by annular features molded or machined into the interior walls of the cylindrical housing that secure the membrane-like filter onto the support as the cylindrical housing is mounted to the funnel.

Although, as discussed above, PEEK is an attractive polymer from which to manufacture the membrane-like filters, other types of polymers and polymer formulations tailored to produce the tear-resistance and wear-resistance of PEEK filters as well as the resistance of PEEK to accumulation of biological tissues, materials, and other solution components, may alternatively be employed in place of, or in addition to, PEEK. These alternative polymers include polycarbonate polymers, polyester polymers, polyamide polymers, and polyvinylidine-floride polymers. Membrane-like filters can be manufactured from combinations of polymers, from polymers embedded in an inorganic or organic material, and from other rigid or compliant films in which tapered apertures can be formed or machined.

Although the present invention has been described in terms of particular embodiments, it is not intended that the invention be limited to these embodiments. Modifications will be apparent to those skilled in the art. For example, membrane-like filters of many different sizes and shapes can be produced as alternative embodiments of the present application. The arrays of micromachined apertures may be square, rectangular, disk-like, or have other such shapes, and may include any of various different numbers of rows and columns of micromachined apertures of various different shapes and sizes. In all cases, the micro-machined apertures are tapered, as discussed with reference to FIGS. 5A-B. The membrane-like filters that represent embodiments of the present application may be machined by laser-drilling processes, in which the angle of light focused through a focusing lens produces the desired taper. Membrane-like filters that represent embodiments of the present application may have thicknesses of 150 μm, 125 μm, 100 μm, 50 μm, 25 μm, or various other thicknesses, depending on cost constraints, requirements for mechanical rigidity, desired flow characteristics, and other such parameters, and may comprise a PEEK film or films or substrates composed of other polymeric materials that resist accumulation of biological tissues, materials, and other solution components during filtration procedures, that resist clogging, and that provides sufficient mechanical strength for particular filtering applications. The tapered apertures in an example filter have widths of approximately six μm and lengths of approximately 40 μm, providing an aperture area of 240 μm. In alternative filters, the tapered apertures may have aperture areas less than 50 μm, between 50 μm and 100 μm, between 100 μm and 150 μm, between 150 μm and 200 μm, or between 200 μm and 250 μm. In certain filters, the dimensions and areas of the apertures may fall within ranges of dimensions and sizes. A pattern of apertures introduced into the membrane-like filter may be grid-like, including grid-like patterns that feature square and rectangular elements as well as grid-like patterns where the axes are not perpendicular and thus produce various types of parallelogram elements, including grids with hexagonal symmetry. Alternatively, the apertures may be densely but randomly positioned, and may be positioned in spiral patterns, patterns of annular rings of increasing radii, or in many other patterns. The taper of the tapered apertures may vary with varying thicknesses of the membrane-like filter and with surface properties of the particular type of material from which the membrane-like filter is made. In certain cases, membrane-like filters can be manufactured from thin, rigid, or semi-rigid inorganic or organic films and inorganic material.

It is appreciated that the previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

1. A membrane-like filer comprising:
a filter that resists accumulation of biological tissues, materials, and other solution components during filtration procedures, that resists clogging, and that provides sufficient mechanical strength to resist wearing and tearing under fluid pressure applied to a circulating-tumor-cell-containing fluid passed through the filter; and
an array of tapered, microscale apertures.

2. The membrane-like filer of claim 1 wherein the filter comprises a polyether ether ketone polymer film that includes an array of tapered, microscale apertures.

3. The membrane-like filer of claim 1 wherein the tapered, microscale apertures of the array of tapered, microscale apertures have aperture areas selected from among:
a range of aperture areas less than 50 μm;
a range of aperture areas of between 50 μm and 100 μm;
a range of aperture areas of between 100 μm and 150 μm;
a range of aperture areas of between 150 μm and 200 μm; and
a range of aperture areas of between 200 μm and 250 μm.

4. The membrane-like filer of claim 1 wherein the filter is composed of one or more of:
polycarbonate polymers;
polyester polymers;
polyamide polymer;
polyvinylidine-floride polymers;
an inorganic compound or substance; and
a small-molecule organic compound or substance.

5. The membrane-like filer of claim 1 wherein the filter has a thickness of one of:
less than 25 μm;
less than 50 μm;
less than 100 μm;
less than 125 μm; and
less than 150 μm.

6. The membrane-like filer of claim 1 wherein the filter has a size and shape designed to cover a porous support within a filter housing or filter holder so that a fluid introduced into the filter housing flows wither through the tapered apertures into the porous support or from the porous support into the tapered apertures, but does not flow around the filter.

7. The membrane-like filer of claim 1 wherein each of the tapered apertures has a larger-area aperture that opens to a first side of the filter and a smaller-area aperture that opens to a second side of the filter, the relative areas of the larger-area apertures and the smaller-area apertures depending on a taper within the tapered apertures and a thickness of the filter.

8. The membrane-like filer of claim 7 wherein the filter is employed to filter a circulating-tumor-cell-containing fluid directed to the first side and passing through the tapered apertures to exit from the second side.

9. A circulating-tumor-cell isolation device comprising:
a first filter-housing component into which a circulating-tumor-cell-containing fluid is directed;
a filter that resists accumulation of biological tissues, materials, and other solution components during filtration procedures, that resists clogging, and that provides sufficient mechanical strength to resist wearing and tearing under fluid pressure applied to a circulating-tumor-cell-containing fluid passed through the filter and that includes an array of tapered, microscale apertures; and
a second filter-housing component that, when coupled with the first filter-housing component, forms a fluid impermeable filtration chamber in which the filter is securely positioned, the filtration chamber comprising a first filtration chamber adjacent to a first face of the filter and a second filtration chamber adjacent to a second side of the filter, with the first filtration chamber in fluid communication with the second filtration chamber through the tapered apertures within the filter.

10. The circulating-tumor-cell isolation device of claim 9 wherein the tapered, microscale apertures of the array of tapered, microscale apertures have aperture areas selected from among:
a range of aperture areas less than 50 μm;
a range of aperture areas of between 50 μm and 100 μm;
a range of aperture areas of between 100 μm and 150 μm;
a range of aperture areas of between 150 μm and 200 μm; and
a range of aperture areas of between 200 μm and 250 μm.

11. The circulating-tumor-cell isolation device of claim 9 wherein the filter is composed of one or more of:
polyether ether ketone polymers;
polycarbonate polymers;
polyester polymers;
polyamide polymer;
polyvinylidine-floride polymers;
an inorganic compound or substance; and
a small-molecule organic compound or substance.

12. The circulating-tumor-cell isolation device of claim 9 wherein the filter has a thickness of one of:
less than 25 μm;
less than 50 μm;
less than 100 μm;
less than 125 μm; and
less than 150 μm.

13. The circulating-tumor-cell isolation device of claim 9 wherein the filter has a size and shape designed to cover a porous support located within the filtration chamber so that a fluid directed through the filtration chamber flows either through the tapered apertures into the porous support or from the porous support into the tapered apertures, but does not flow around the filter.

14. The circulating-tumor-cell isolation device of claim 9
wherein each of the tapered apertures has a larger-area aperture that opens to the first side of the filter and a smaller-area aperture that opens to the second side of the filter, the relative areas of the larger-area apertures and the smaller-area apertures depending on a taper within the tapered apertures and a thickness of the filter; and
wherein the filter is employed to filter a circulating-tumor-cell-containing fluid directed to the first side and passing through the tapered apertures to exit from the second side.

15. A method for isolating circulating tumor cells, the method comprising:
preparing a circulating-tumor-cell-containing fluid;
passing the circulating-tumor-cell-containing fluid through a filter that resists accumulation of biological tissues, materials, and other solution components during filtration procedures, that resists clogging, and that provides sufficient mechanical strength to resist wearing and tearing under fluid pressure applied to the circulating-tumor-cell-containing fluid passed through the filter and that includes an array of tapered, microscale apertures; and
staining the CTC cells remaining on a surface of the filter and examining the CTC sells under a microscope to identify, and count, and characterize the CTCs or flushing the CTCs from the filer into an analytical solution and analyzing the analytical solution to count and characterize the CTCs.

16. The method of claim 17 wherein the tapered, microscale apertures of the array of tapered, microscale apertures have aperture areas selected from among:
a range of aperture areas less than 50 μm;
a range of aperture areas of between 50 μm and 100 μm;
a range of aperture areas of between 100 μm and 150 μm;
a range of aperture areas of between 150 μm and 200 μm; and
a range of aperture areas of between 200 μm and 250 μm.

17. The method of claim 17 wherein the filter is composed of one or more of:
polyether ether ketone polymers;
polycarbonate polymers;
polyester polymers;
polyamide polymer;
polyvinylidine-floride polymers;
an inorganic compound or substance; and
a small-molecule organic compound or substance.

18. The method of claim 17 wherein the filter has a thickness of one of:
less than 25 μm;
less than 50 μm;
less than 100 μm;
less than 125 μm; and
less than 150 μm.

19. The method of claim 17 wherein the filter has a size and shape designed to cover a porous support within a filter housing or filter holder so that a fluid introduced into the filter housing flows either through the tapered apertures into the porous support or from the porous support into the tapered apertures, but does not flow around the filter.

20. The method of claim 17
wherein each of the tapered apertures has a larger-area aperture that opens to a first side of the filter and a smaller-area aperture that opens to a second side of the filter, the relative areas of the larger-area apertures and the smaller-area apertures depending on a taper within the tapered apertures and a thickness of the filter; and
wherein the filter is employed to filter a circulating-tumor-cell-containing fluid directed to the first side and passing through the tapered apertures to exit from the second side.
